(19) United States
(12) Patent Application Publication
(10) Pub. No.: US 2025/0011292 A1
(43) Pub. Date: Jan. 9, 2025

(54) NOVEL RHODANINE DERIVATIVES AND USES THEREOF

(71) Applicant: GEORGIA STATE UNIVERSITY RESEARCH FOUNDATION, INC., Atlanta, GA (US)

(72) Inventors: Binghe WANG, Marietta, GA (US); Xiaoxiao Yang, Marietta, GA (US); Ming Luo, Atlanta, GA (US); Mukesh Kumar, Decatur, GA (US)

(73) Assignee: GEORGIA STATE UNIVERSITY RESEARCH FOUNDATION, INC., Atlanta, GA (US)

(21) Appl. No.: 18/698,251

(22) PCT Filed: Oct. 12, 2022
(86) PCT No.: PCT/US2022/046418

§ 371 Date: Apr. 3, 2024

Related U.S. Application Data
(60) Provisional application No. 63/262,518, filed on Oct. 14, 2021.

(51) Int. Cl. C07D 277/36; A61K 31/426; A61K 31/427; A61K 31/4439; A61K 31/496; A61K 31/5377; A61P 31/14; C07D 417/06; C07D 417/14

(52) U.S. Cl. CPC C07D 277/36; A61K 31/426; A61K 31/427; A61K 31/4439; A61K 31/496; A61K 31/5377; A61P 31/14; C07D 417/06; C07D 417/14

(57) ABSTRACT

Novel rhodanine derivatives and methods for their use are provided herein. The rhodanine derivatives are useful in treating or preventing viral infections, including coronavirus infections (e.g., a SARS-CoV-2 virus infection). The rhodanine derivatives are also useful as photosensitizers for photodynamic therapy in treating or preventing cancer in a subject.

### CROSS-REFERENCE TO PRIORITY APPLICATION

This application claims priority to U.S. Provisional Application No. 63/262,518, filed Oct. 14, 2021, which is incorporated herein by reference in its entirety.

### BACKGROUND

Coronaviruses are a group of related viruses that cause diseases in mammals and birds. In humans, coronaviruses cause respiratory tract infections that can be mild, such as some cases of the common cold. Other respiratory tract infections caused by coronaviruses that can be lethal to humans include Severe Acute Respiratory Syndrome (SARS), Middle East Respiratory Syndrome (MERS), and 2019 Novel Coronavirus (COVID-19), which is responsible for the pandemic of 2020.

### SUMMARY

Described herein are novel rhodanine derivatives and methods for their use. The rhodanine derivatives described herein are useful in treating or preventing viral infections, including coronavirus infections (e.g., a SARS-CoV-2 virus infection). The rhodanine derivatives described herein are also useful as photosensitizers for photodynamic therapy in, for example, treating or preventing cancer in a subject.

Rhodanine derivatives as described herein include compounds of the following formula:

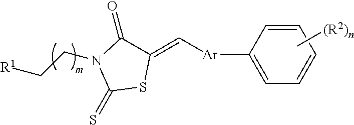

and pharmaceutically acceptable salts or prodrugs thereof. In these compounds, m is 1, 2, 3, 4, or 5; n is 0, 1, 2, 3, 4, or 5; Ar is substituted or unsubstituted aryl or substituted or unsubstituted heteroaryl; R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and each R² is independently halogen, hydroxyl, trifluoroalkyl, substituted or unsubstituted amino, substituted or unsubstituted alkoxy, or substituted or unsubstituted alkyl. Optionally, Ar is unsubstituted aryl (e.g., phenyl) or unsubstituted heteroaryl (e.g., furanyl). In some cases, R¹ is methyl, morpholinyl, morpholinylmethyl, morpholinylethyl, thiomorpholinyl, pyridinyl, or pyridinylmethyl. Optionally, n is 1, 2, 3, 4, or 5. In some cases, each R² is independently selected from the group consisting of fluoro, hydroxyl, alkoxy, and trifluoromethyl. Optionally, n is 0. Optionally, m is 1.

In some examples, the compound is selected from the group consisting of:

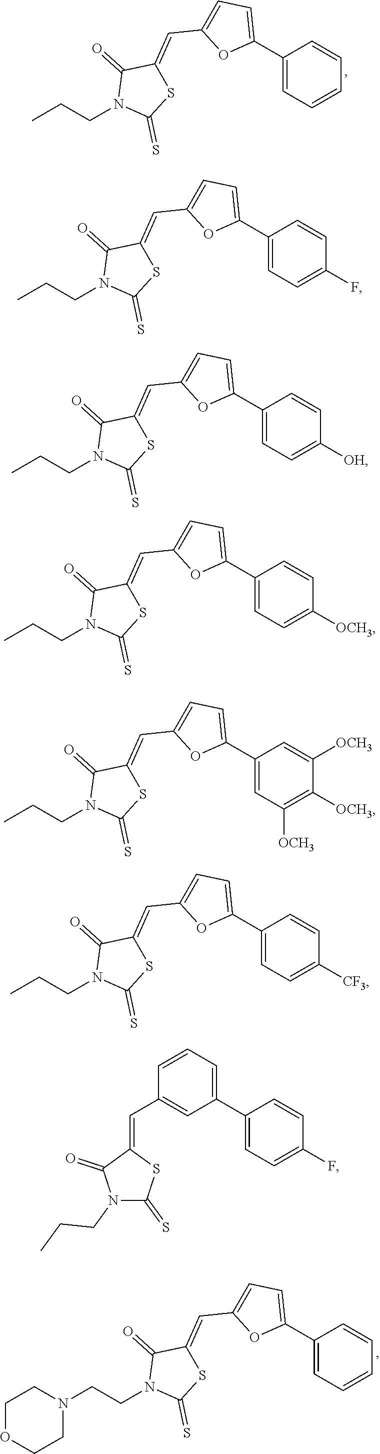

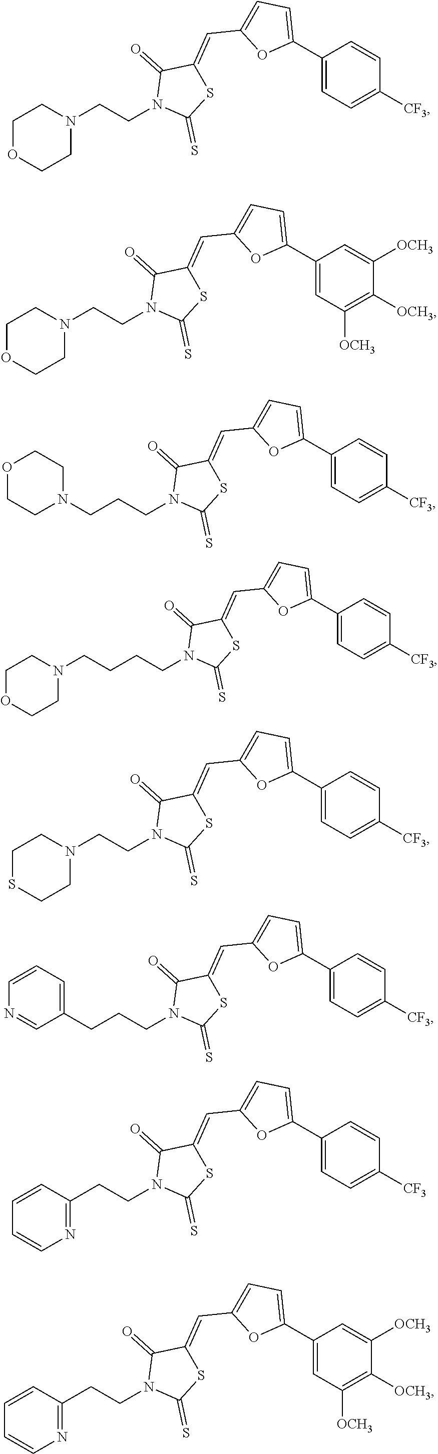

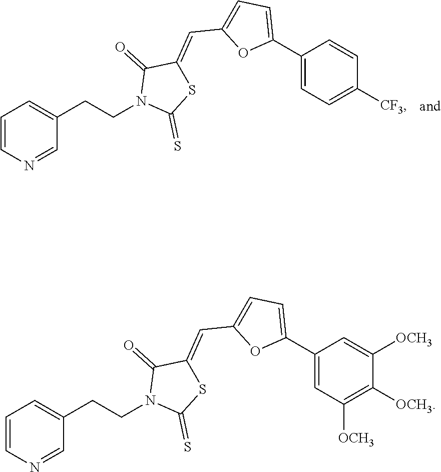 [SMILES: O=C1/C(=C/c2ccc(-c3ccc(C(F)(F)F)cc3)o2)SC(=S)N1CCc1cccnc1 ; COc1cc(-c2ccc(/C=C3\SC(=S)N(CCc4cccnc4)C3=O)o2)cc(OC)c1OC]

Also described herein are pharmaceutical compositions comprising a compound as described herein and a pharmaceutically acceptable carrier.

Further described herein is a kit comprising a compound or a pharmaceutical composition as described herein.

Methods of treating or preventing a viral infection in a subject are also described herein. A method of treating or preventing a viral infection in a subject comprises administering to the subject an effective amount of a compound or a pharmaceutical composition as described herein. Optionally, the viral infection is a coronavirus infection (e.g., a SARS-CoV-2 virus infection). In some cases, the method can further comprise administering to the subject a second therapeutic agent (e.g., an antiviral compound).

Methods of inhibiting viral replication in a cell are also described herein. A method of inhibiting viral replication in a cell comprises contacting a cell with an effective amount of a compound as described herein. Optionally, the cell is infected with a coronavirus (e.g., a SARS-CoV-2 virus). The contacting can be performed in vitro or in vivo.

Further described herein are methods of treating or preventing cancer in a subject, comprising administering to the subject an effective amount of a compound or a pharmaceutical composition as described herein, and applying energy to the subject at a wavelength capable of activating the compound or the pharmaceutical composition.

The details of one or more embodiments are set forth in the drawings and the description below. Other features, objects, and advantages will be apparent from the description and drawings, and from the claims.

### DESCRIPTION OF THE DRAWINGS

FIGS. 1A-1C are graphs showing the inhibition of SARS-CoV-2 replication in Huh-7 cells by BW-PS-108 (FIG. 1A), BW-PS-109 (FIG. 1B), and Compound 136 (FIG. 1C). The top line in FIGS. 1A and 1B represent cytotoxicity. Cell pellets from infected cells were collected at 48 hours after infection and viral RNA levels were measured by RT-PCR.

FIG. 1D is a graph showing the activity of BW-PS-119 against the SARS-CoV-2 virus in C57BL/6 mice. Each data point represents an individual mouse.

FIGS. 2A-2K are graphs showing the dose response of compounds as described herein in HeLa cells. The compounds tested include BW-PS-101 (FIG. 2A), BW-PS-102 (FIG. 2B), BW-PS-103 (FIG. 2C), BW-PS-104 (FIG. 2D), BW-PS-105 (FIG. 2E), BW-PS-106 (FIG. 2F), BW-PS-107 (FIG. 2G), BW-PS-108 (FIG. 2H), BW-PS-109 (FIG. 2I), BW-PS-110 (FIG. 2J), and cisplatin as a control (FIG. 2K).

FIGS. 3A-3K are graphs showing the dose response of compounds as described herein in MCF-7 cells. The compounds tested include BW-PS-101 (FIG. 3A), BW-PS-102 (FIG. 3B), BW-PS-103 (FIG. 3C), BW-PS-104 (FIG. 3D), BW-PS-105 (FIG. 3E), BW-PS-106 (FIG. 3F), BW-PS-107 (FIG. 3G), BW-PS-108 (FIG. 3H), BW-PS-109 (FIG. 3I), BW-PS-110 (FIG. 3J), and cisplatin as a control (FIG. 3K). The cells were treated and allowed the incubate for 24 hours (labeled in the figures as the compound name followed by “DARK”) or were treated, incubated for 30 minutes, exposed to light for a designated time, and then incubated for 24 hours (labeled in the figures as the compound name followed by “EXPO”).

FIG. 4 is a graph showing the cell viability of the vehicle-treated (0.5% DMSO) HeLa cells, absorbance at 450 nm. The experiment was performed in triplicate. (ns: no significance)

FIG. 5 is an ultraviolet (UV) spectrum of BW-PS-101.

FIG. 6 is a graph showing the cell viability of the vehicle treated (0.5% DMSO) HeLa cells. The cell viabilities under darkness were set to be 100%. The experiments were repeated in triplicate. (ns: no significance)

FIG. 7 is a dose-response curve of BW-PS-101 in HeLa cells under different light irradiation energy. The experiments were repeated in triplicate, data presented in mean±SD).

FIG. 8 are fluorescence microscopy images of BW-PS-105 (first row), BW-PS-110 (second row), and BW-PS-120 (third row) localized in early endosomes. The left panel of each row shows the endosome GFP label (GFP channel), the middle panel of each row shows the compound, and the right panel of each row shows the merged image of the respective left and middle panels.

### DETAILED DESCRIPTION

Described herein are novel rhodanine derivatives and methods for their use. The rhodanine derivatives described herein are useful in treating or preventing viral infections, including coronavirus infections (e.g., a SARS-CoV-2 virus infection). The rhodanine derivatives described herein are also useful as photosensitizers for photodynamic therapy in treating or preventing cancer in a subject.

### I. Compounds

A class of rhodanine compounds described herein is represented by Formula I:

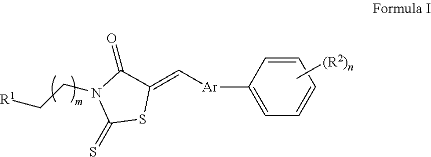

and pharmaceutically acceptable salts or prodrugs thereof.

In Formula I, m is 1, 2, 3, 4, or 5. In some cases, m is 1.

Also in Formula I, n is 0, 1, 2, 3, 4, or 5. In some cases, n is 0. In some cases, n is 1. In some cases, n is 2. In some cases, n is 3.

Additionally in Formula I, Ar is substituted or unsubstituted aryl or substituted or unsubstituted heteroaryl. Optionally, Ar is phenyl or furanyl.

Further in Formula I, R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Optionally, R¹ is methyl, morpholinyl, morpholinylmethyl, morpholinylethyl, thiomorpholinyl, pyridinyl, or pyridinylmethyl. Optionally, R¹ is not alkenyl.

Additionally in Formula I, each R² is independently halogen, hydroxyl, trifluoroalkyl, substituted or unsubstituted amino, substituted or unsubstituted alkoxy, or substituted or unsubstituted alkyl. Optionally, each R² is independently selected from the group consisting of fluoro, hydroxyl, alkoxy, and trifluoromethyl. Optionally, R² is not a substituted carbonyl.

Optionally, when m is 2, R¹ is morpholinyl, Ar is furanyl, and n is 1, then R² is not p-chloro.

Examples of Formula I include the following compounds:

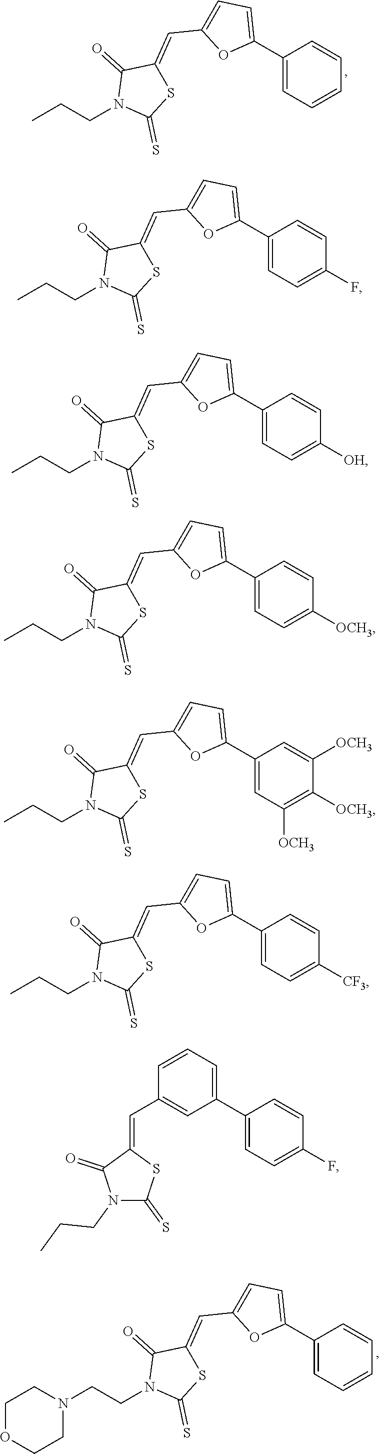

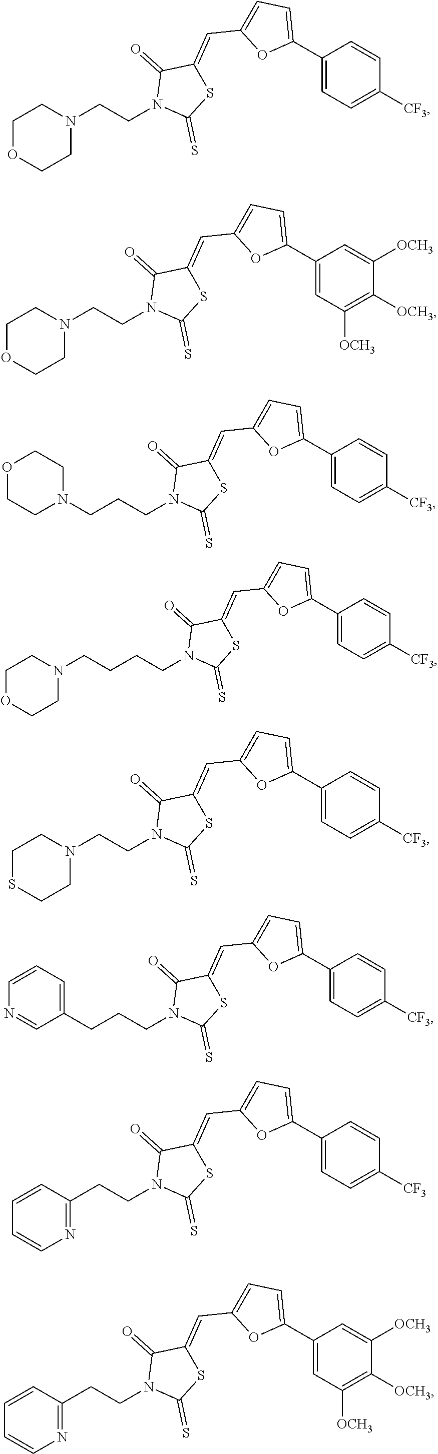

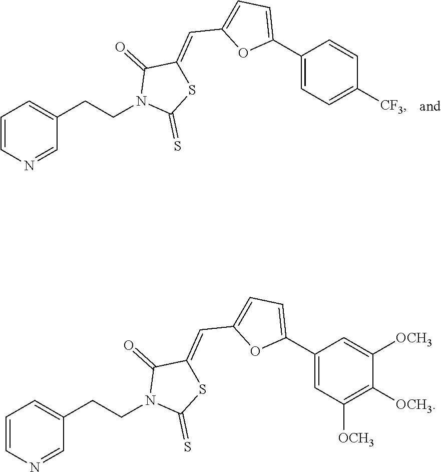 [SMILES: O=C1/C(=C/c2ccc(-c3ccc(C(F)(F)F)cc3)o2)SC(=S)N1CCc1cccnc1 ; COc1cc(-c2ccc(/C=C3\SC(=S)N(CCc4cccnc4)C3=O)o2)cc(OC)c1OC]

In some cases, the compound is not

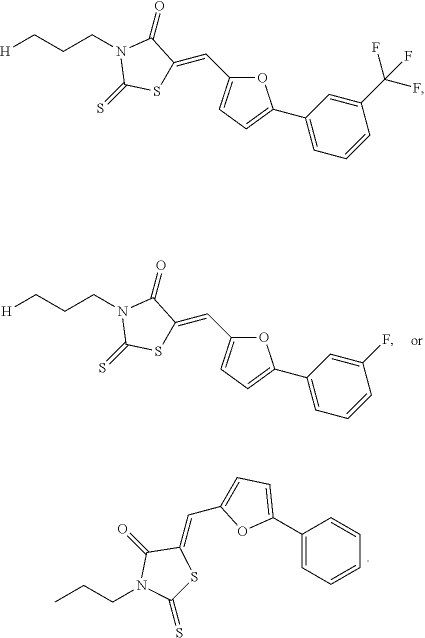 [SMILES: CCCN1C(=O)/C(=C/c2ccc(-c3cccc(C(F)(F)F)c3)o2)SC1=S ; CCCN1C(=O)/C(=C/c2ccc(-c3cccc(F)c3)o2)SC1=S ; CCCN1C(=O)/C(=C/c2ccc(-c3ccccc3)o2)SC1=S]

As used herein, the terms alkyl, alkenyl, and alknyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alky, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₅-C₁₂ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀, heterocycloalkyl, C₃-C₂, heterocycloalkenyl, and C₃-C₂₀ heterocycloalkynyl. Additional ranges of these groups useful with the compounds and methods described herein include C₃-C₂₀ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imidazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryoloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, cycloalkoxy, aryloxy, amino, alkyl, alkenyl, alkynyl, airy, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e g., linear decane (—(CH₂)₉—CH₃).

### II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure, Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Generally, the compounds described herein can be prepared according to the methods depicted below in Scheme 1 and Scheme 2.

Rhodanine is synthesized by reacting amine with carbon disulfide and chloroacetic acid in a sequential manner. The aryl furane aldehyde is synthesized by Suzuki coupling aryl halide with formylfuran boronic acid. The final compound is synthesized by aldol condensation between the rhodanine 1 and the aldehyde 2.

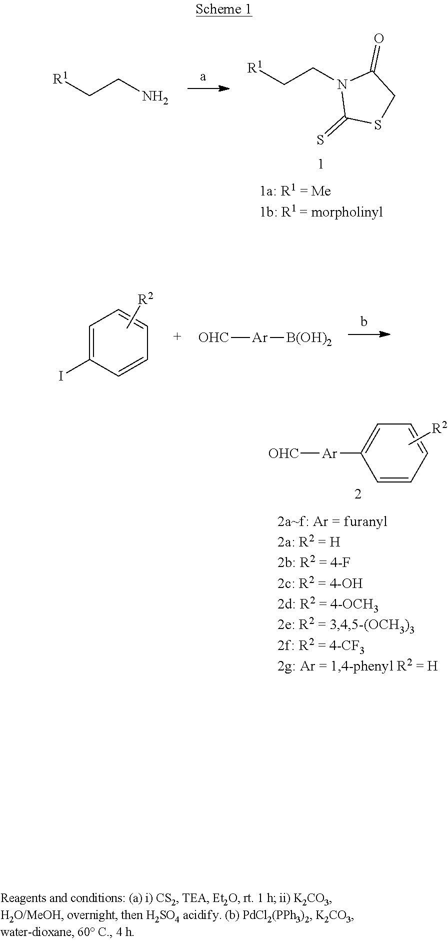

TABLE 1

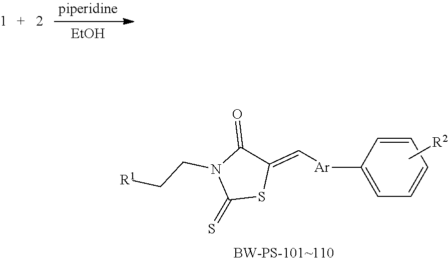

| BW-PS | | | |
|---|---|---|---|
| NO. | R¹ | Ar | R² |
| 101 | Me | 2,5-furanyl | H |
| 102 | Me | 2,5-furanyl | 4-F |
| 103 | Me | 2,5-furanyl | 4-OH |
| 104 | Me | 2,5-furanyl | 4-OCH₃ |
| 105 | Me | 2,5-furanyl | 3,4,5- |
| | | | (OCH₃)₃ |
| 106 | Me | 2,5-furanyl | 4-CF₃ |
| 107 | Me | 1,3-phenyl | 4-F |
| 108 | morpholinyl | 2,5-furanyl | H |
| 109 | morpholinyl | 2,5-furanyl | 4-CF₃ |
| 110 | morpholinyl | 2,5-furanyl | 3,4,5- |
| | | | (OCH₃)₃ |

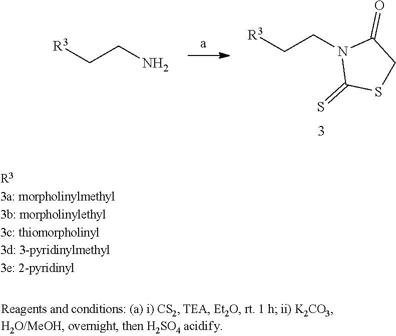

TABLE 2

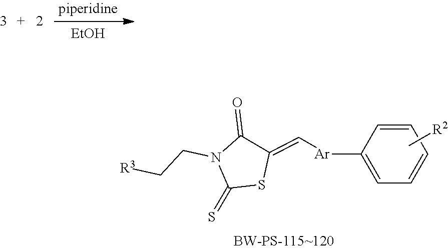

| BW-PS | | | |
|---|---|---|---|
| NO. | R³ | Ar | R² |
| 115 | morpholinylmethyl | 2,5-furanyl | 4-CF₃ |
| 116 | morpholinylethyl | 2,5-furanyl | 4-CF₃ |
| 117 | thiomorpholinyl | 2,5-furanyl | 4-CF₃ |
| 118 | 3-pyridinylmethyl | 2,5-furanyl | 4-CF₃ |
| 119 | 2-pyridinyl | 2,5-furanyl | 4-CF₃ |
| 120 | 2-pyridinyl | 2,5-furanyl | 3,4,5- |
| | | | (OCH₃)₃ |

Exemplary methods for synthesizing the compounds as described herein are provided in Example 1 below.

### III. Pharmaceutical Formulations

The compounds described herein or derivatives thereof can be provided in a pharmaceutical composition. The one or more compounds described herein can be provided as pharmaceutical compositions administered in combination with one or more other therapeutic or prophylactic agents. As used throughout, a therapeutic agent is a compound or composition effective in ameliorating a pathological condition. Illustrative examples of therapeutic agents include, but are not limited to, anti-viral agents, anti-opportunistic agents, antibiotics, and immunostimulatory agents. Optionally, more than one therapeutic agent is administered in combination with the provided compositions.

The one or more compounds described herein, with or without additional agents, can be provided in the form of an inhaler or nebulizer for inhalation therapy. As used herein, inhalation therapy refers to the delivery of a therapeutic agent, such as the compounds described herein, in an aerosol form to the respiratory tract (i.e., pulmonary delivery). As used herein, the term aerosol refers to very fine liquid or solid particles carried by a propellant gas under pressure to a site of therapeutic application. When a pharmaceutical aerosol is employed, the aerosol contains the one or more compounds described herein, which can be dissolved, suspended, or emulsified in a mixture of a fluid carrier and a propellant. The aerosol can be in the form of a solution, suspension, emulsion, powder, or semi-solid preparation. In the case of a powder, no propellant gas is required when the device is a breath activated dry powder inhaler. Aerosols employed are intended for administration as fine, solid particles or as liquid mists via the respiratory tract of a patient.

The propellant of an aerosol package containing the one or more compounds described herein can be capable of developing pressure within the container to expel the compound when a valve on the aerosol package is opened. Various types of propellants can be utilized, such as fluorinated hydrocarbons (e.g., trichloromonofluormethane, dichlorodifluoromethane, and dichlorotetrafluoroethane) and compressed gases (e.g., nitrogen, carbon dioxide, nitrous oxide, or Freon). The vapor pressure of the aerosol package can be determined by the propellant or propellants that are employed. By varying the proportion of each component propellant, any desired vapor pressure can be obtained within the limits of the vapor pressure of the individual propellants.

As described above, the one or more compounds described herein can be provided with a nebulizer, which is an instrument that generates very fine liquid particles of substantially uniform size in a gas. The liquid containing the one or more compounds described herein can be dispersed as droplets about 5 mm or less in diameter in the form of a mist. The small droplets can be carried by a current of air or oxygen through an outlet tube of the nebulizer. The resulting mist can penetrate into the respiratory tract of the patient.

Additional inhalants useful for delivery of the compounds described herein include intra-oral sprays, mists, metered dose inhalers, and dry powder generators (See Gonda, J. Pharm. Sci. 89:940-945, 2000, which is incorporated herein by reference in its entirety, at least, for inhalation delivery methods taught therein). For example, a powder composition containing the one or more compounds as described herein, with or without a lubricant, carrier, or propellant, can be administered to a patient. The delivery of the one or more compounds in powder form can be carried out with a conventional device for administering a powder pharmaceutical composition by inhalation.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd Edition, Loyd et al. eds., Pharmaceutical Press and Philadelphia College of Pharmacy at University of the Sciences (2012) Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN@ (ICI Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminium monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame oil, glycerol, tetrahydrofurfuryl alcohol, polyethylene glycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethylene glycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and lauryl sulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0,0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg, Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IV. Methods of Use

Provided herein are methods to treat or prevent a viral infection in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which a viral replication is inhibited in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating or preventing viral infections in humans, including, without limitation, pediatric and geriatric populations, and in animals, e.g., veterinary applications.

In some examples, the viral infection is a coronavirus infection (e.g., a SARS-CoV-2 virus infection).

Also provided herein are methods to treat or prevent cancer in a subject. The methods include administering to a subject an effective amount of one or more of the compounds or compositions described herein, or a pharmaceutically acceptable salt or prodrug thereof. Optionally, the cancer is bladder cancer, brain cancer, breast cancer (e.g., triple negative breast cancer), bronchus cancer, colorectal cancer (e.g., colon cancer, rectal cancer), cervical cancer, chondrosarcoma, endometrial cancer, gastrointestinal cancer, gastric cancer, genitourinary cancer, glioblastoma, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer.

The methods of treating or preventing a viral infection or cancer, as described herein, can further comprise administering to the subject a second compound, biomolecule, or composition. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

In the treatment of viral infections, the second compound or composition can include an antiviral compound or mixtures of antiviral compounds (e.g., pegylated interferon-α, ribavirin, and mixtures thereof). Antiviral compounds that can be used in combination with the compounds described herein include, for example, nucleoside polymerase inhibitors, non-nucleoside polymerase inhibitors, protease inhibitors, nucleoside or nucleotide reverse transcriptase inhibitors, non-nucleoside reverse transcriptase inhibitors, entry inhibitors, assembly inhibitors, integrase inhibitors, kinase inhibitors, enzyme inhibitors, maturation inhibitors, M2 inhibitors, and neuraminidase inhibitors. Examples of such additional antiviral compounds include, but are not limited to abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir (Tamiflu), peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, and/or zidovudine.

In the treatment of cancer, the second compound or composition can include abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

The rhodanine compounds described herein are also useful as photosensitizers for photodynamic therapy in, for example, treating or preventing cancer in a subject. Optionally, the methods of treating cancer include applying energy to the subject at a wavelength capable of activating the compound or the pharmaceutical composition. For example, the methods can include applying a light source for irradiating light to the desired treatment site of the subject. The light has a waveband that corresponds, partially or fully, to the characteristic light absorption waveband of the rhodanine compound. In some cases, the compounds described herein can be used as endosome probes to specifically target and label the endosome.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a viral infection or cancer), during early onset (e g., upon initial signs and symptoms of a viral infection or cancer, or after the development of a viral infection or cancer. Prophylactic administration can occur for several days to years prior to the manifestation of symptoms of a viral infection or cancer. Therapeutic treatment involves administering to a subject a therapeutically effective amount of the compounds and compositions or pharmaceutically acceptable salts thereof as described herein after a viral infection or cancer is diagnosed.

The methods of treating or preventing a viral infection or cancer can also include administering the compounds or pharmaceutical compositions described herein by one or more clinically acceptable routes. The compounds or pharmaceutical compositions described herein can be administered orally, intraperitoneally, sublingually, subcutaneously, intravenously, or any clinically acceptable administration route.

The compounds described herein are also useful for inhibiting viral replication in a cell are also described herein. The methods for inhibiting viral replication in a cell include contacting a cell with an effective amount of a compound as described herein. In some cases, the compounds described herein are useful for modulating the effects of endosomes and/or lysosomes (e.g., by accumulating within endosomes and/or lysosomes), thus impairing viral release from the endosomes and/or lysosomes. Optionally, the cell is infected with a coronavirus (e.g., a SARS-CoV-2 virus). The contacting can be performed in vitro or in vivo.

### V. Kits

Also provided herein are kits for treating or preventing a viral infection in a subject or for treating or preventing cancer in a subject. A kit can include any of the compounds or compositions described herein. For example, a kit can include one or more compounds of Formula I. A kit can further include one or more additional agents, such as a second compound, biomolecule, or composition (e.g., one or more anti-viral agents or anti-cancer agents). A kit can include an oral formulation of any of the compounds or compositions described herein. A kit can include an intravenous formulation of any of the compounds or compositions described herein. A kit can also include an energy source (e.g., a light source for irradiating a light beam). A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10% 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., rate of viral replication or size of a tumor or rate of tumor growth) of the disease in a subject as compared to a control. As used herein, control refers to the untreated condition (e.g., at-risk populations not treated with the compounds and compositions described herein or tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; nonhuman primates, e.g., apes and monkeys; cattle; horses; sheep; rats; nice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Compound Synthesis

General information: All starting materials were purchased from Sigma-Aldrich or Oakwood. Analytical-grade solvents from Sigma-Aldrich were used for all reactions. Anhydrous solvents were used for all moisture-sensitive reactions. ¹H (400 MHz) and ¹³C NMR (100 MHz) were obtained on a Bruker Avance 400 NMR spectrometer in deuterated solvent from Cambridge Isotope Laboratories, Inc. Chemical shifts were reported as 6 values (ppm). TMS (δ=0.00 ppm) or residual peaks of the deuterated solvent were used as the internal reference.

### Synthesis 3-propyl-2-thioxothiazolidin-4-one (1a)

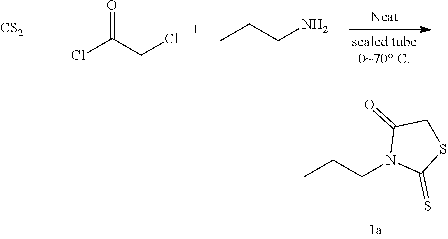

Carbon disulphide (50 mmol) and propyl amine (10 mmol) were mixed together in a sealed tube, and the mixture was cooled down to 0° C. Then, chloroacetyl chloride (10 mmol) was added and the tube was closed. The mixture was then heated to 70° C. for about 2 hours. After which the reaction temperature was lowered. TLC showed consumption of the starting material (propyl amine). Then, the mixture was diluted with dichloromethane and washed successively water (200 ml), brine (200 ml) and the organic layer was dried and anhydrous sodium sulphate. The crude product was purified by silica gel column chromatography (Hexane/Ethyl Acetate: 4/1) to afford a pure product 1a as dark brownish oil with an isolated yield of 31%. ¹H NMR (CDCl₃) δ 3.99 (s, 2H), 3.98-3.92 (m, 2H), 1.73-1.61 (m, 2H), 0.95 (t, J=7.5 Hz, 3H). ¹³C NMR (CDCl₃) δ 201.33, 173.92, 46.21, 35.37, 20.17, 11.20. HRMS (ESI) m/z: Calculated for C₆H₉NOS₂Na [M+Na]⁺ 198.0014; Found 198.0023.

### Synthesis of 3-(2-morpholinoethyl)-2-thioxothiazolidin-4-one (1b)

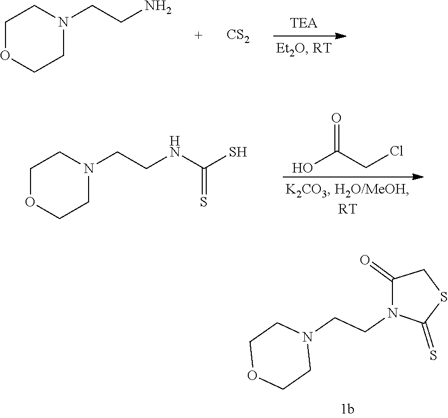 [SMILES: NCCN1CCOCC1 ; "CS2" ; S=C(S)NCCN1CCOCC1 ; O=C(O)CCl ; O=C1CSC(=S)N1CCN1CCOCC1]

In a dry 100 ml round bottom flask equipped with stirring bar, 1 g (7.68 mmol) N-aminoethylmorpholine was added followed by 10 ml dry Et₂O and 1.06 ml triethylamine (7.68 ml). Then, 0.584 g (7.68 mmol) carbon disulfide in 2 ml dry Et₂O was added via injection. The reaction was stirred at room temperature for 1 h, at which the color of the reaction became yellow. The reaction mixture was concentrated by rotavap under reduced pressure, and the residue was dissolved in 15 ml of the mixture of water:methanol=4:1 (v:v). 0.53 g (3.84 mmol) K₂CO₃ was added as powder followed by addition of 0.73 g (7.68 mmol) chloroacetate dissolved in 4 ml water. The reaction was then stirred at room temperature overnight. After conformation with TLC (DCM:MeOH=20:1, v:v) of the formation of the product (Rf=0.45) and the hydrated form of the product (Rf=0.3), the reaction mixture was acidified with 2 M H₂SO₄ then stirred for another 12 h. TLC showed conversion of the hydrated intermediate to the final product. Then MeOH in the reaction mixture was removed by rotavap and the aqueous layer was washed with Et₂O (50 ml×2). The aqueous layer was basified with NaHCO₃ powder and extracted with EtOAc (50 ml×2). The combined organic layer was washed with saturated NaHCO₃ and brine for three times then dried over Na₂SO₄. Concentrated in vacuo followed by purification with silica flash column (DCM:MeOH=20:1-10:1, v:v) gave the final product as a yellowish-red semisolid with a yield of 1.0 g (53%). ¹H NMR (400 MHz, CDCl₃) δ 4.05 (t, J=6.7 Hz, 2H), 3.93 (s, 2H), 3.56 (t, J=4.4 Hz, 4H), 2.55 (t, J=6.7 Hz, 2H), 2.46 (t, J=4.4 Hz, 4H).

### Synthesis of N-substituted rhodanine derivative 3a-e

The synthesis of each of compounds 3a-e was performed analogously to the synthesis of 1b.

N-morpholinopropylrhodanine (3a): Red solid. Yield: 856 mg (47%). ¹H NMR (400 MHz, Acetone) δ 4.19 (s, 2H), 4.04 (t, J=7.2 Hz, 2H), 3.59 (t, J=4.4 Hz, 4H), 2.39-2.33 (m, 6H), 1.86-1.75 (m, 2H). ¹³C NMR (101 MHz, Acetone) δ 203.00, 174.32, 66.61, 56.05, 53.66, 42.94, 35.32, 22.74.

N-morpholinobutylrhodanine (3b): Orange solid. Yield: 412 mg (43%). ¹H NMR (400 MHz, Acetone d6) δ 4.20 (s, 2H), 3.97 (t, J=7.6 Hz, 2H), 3.58 (t, J=4.4 Hz, 4H), 2.35-2.30 (m, J, 6H), 1.72-1.59 (m, 2H), 1.57-1.42 (m, 2H). ¹³C NMR (101 MHz, Acetone-d6) δ 203.04, 174.21, 66.58, 58.00, 53.68, 44.12, 35.23, 24.42, 23.52.

N-thiomorpholinoethylrhodanine (3c): Yield: 413 mg (46%). ¹³C NMR (101 MHz, Acetone) δ 203.00, 174.20, 55.15, 54.43, 41.36, 35.15, 27.68.

N-(3-pyridinyl)propylrhodanine (3d): Red semi-solid, yield: 490 mg (53%). ¹H NMR (400 MHz, CDCl₃) δ 8.52 (d, J=6.0 Hz, 2H), 7.23 (d, J=6.0 Hz, 2H), 4.04 (t, J=7.7 Hz, 2H), 3.91 (s, 1H), 2.73 (t, J=7.7 Hz, 2H), 2.09-1.99 (m, 2H). ¹³C NMR (101 MHz, CDCl₃) δ 201.14, 173.83, 151.53, 148.29, 124.11, 44.05, 35.34, 32.43, 26.48.

N-(2-pyridinyl)ethylrhodanine (3e): Red semi-solid, yield: 650 mg (67%). ¹H NMR (400 MHz, CDCl₃) δ 8.51 (dd, J=4.9, 0.8 Hz, 1H), 7.61 (td, J=7.7, 1.8 Hz, 1H), 7.19 (d, J=7.8 Hz, 1H), 7.17-7.13 (m, 1H), 4.36 (dd, J=8.1, 7.0 Hz, 2H), 3.95 (s, 2H), 3.16-3.08 (m, 2H). ¹³C NMR (101 MHz, CDCl₃) δ 201.07, 173.63, 157.59, 149.15, 136.88, 123.59, 121.94, 44.12, 35.36, 34.58.

### General Procedure for the Synthesis of 2a-f

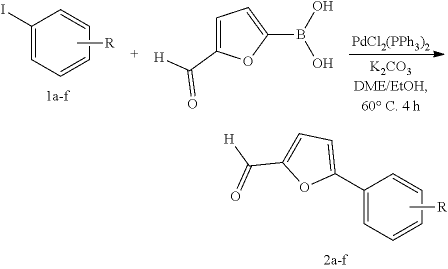

R=a:H; b:4-F; c: 4-OH; d:4-OCH₃; e: 3,4,5-(OCH₃)₃; f: 4-CF₃

Compound 1a-f (1.35 mmol) and (3-formylphenyl)boronic acid (1.62 mmol) were added to a 20 ml vial, followed by K₂CO₃ (1.62 mmol) and 4 ml of solvent (DME/Ethanol/Water-1:0.5:1) was added under argon. Then, 0.07 mmol of PdCl₂(PPh₃)₂ was added and the mixture was stirred at 65° C. for about 4 h and stirred overnight at room temperature. At the consumption of the starting material, the solvent was removed in vacuo and the residue diluted with dichloromethane and washed successively with dilute hydrochloric acid (0.5M, 100 ml×3), saturated sodium bicarbonate (100 ml×3), brine (100×1) and the organic layer was dried and anhydrous sodium sulphate. The crude product was purified by silica gel column chromatography (DCM only) to afford a pure product 2a-f.

### 5-phenylfuran-2-carbaldehyde (2a)

Colorless oil with an isolated yield of 65% ¹H NMR (CDCl₃) δ 9.64 (s, 1H), 7.84-7.77 (m, 2H), 7.50-7.34 (m, 3H), 7.31 (d, J=3.8 Hz, 1H), 6.83 (d, J=3.7 Hz, 1H). ¹³C NMR (CDCl₃) δ 177.24, 159.42, 152.02, 129.71, 128.97, 125.30, 107.74. HRMS (ESI) m/z: Calculated for C₁₁H₈O₂Na [M+Na]f 195.0422; Found 195.0426.

### 5-(4-fluorophenyl)furan-2-carbaldehyde (2b)

Off-white solid with an isolated yield of 74% ¹H NMR (CDCl₃) δ 9.65 (s, 1H), 7.82 (dd, J=8.6, 5.3 Hz, 2H), 7.33 (d, J=3.7 Hz, 1H), 7.15 (t, J=8.6 Hz, 2H), 6.80 (d, J=3.8 Hz, 1H). ¹³C NMR (CDCl₃) δ 177.14, 164.76, 162.26, 158.50, 152.05, 127.37, 127.29, 125.39, 125.35, 123.71, 116.30, 116.08, 107.41, 107.39. HRMS (ESI) m/z: Calculated for C₁₁H₇FO₂Na [M+Na]⁺213.0328; Found 213.0335.

### 5-(4-hydroxyphenyl)furan-2-carbaldehyde (2c)

Off-white solid with an isolated yield of 70% ¹H NMR (DMSO-d6) δ 10.17 (s, 1H), 9.51 (s, 1H), 7.74-7.66 (m, 2H), 7.60 (d, J=3.8 Hz, 1H), 7.04 (d, J=3.8 Hz, 1H), 6.93-6.85 (m, 2H). ¹³C NMR (DMSO-d6) δ 177.63, 159.74, 159.52, 151.43, 127.42, 120.22, 116.53, 107.05. HRMS (ESI) m/z: Calculated for C₁₁H₇O₃ [M−H]⁻ 187.0398; Found 187.0395.

### 5-(4-methoxyphenyl)furan-2-carbaldehyde (2d)

Off-white solid with an isolated yield of 88% ¹H NMR (CDCl₃) δ 9.60 (s, 1H), 7.76 (d, J=8.8 Hz, 2H), 7.30 (d, J=3.7 Hz, 1H), 7.00-6.92 (m, 2H), 6.71 (d, J=3.7 Hz, 1H), 3.85 (s, 3H). ¹³C NMR (CDCl₃) δ 176.83, 160.89, 159.81, 151.61, 126.98, 121.78, 114.43, 106.33, 55.40. HRMS (ESI) m/z: Calculated for C₁₂H₁₁O₃Na [M+Na]⁺225.0528; Found 225.0534.

### 5-(3,4,5-trimethoxyphenyl)furan-2-carbaldehyde (2e)

Off-white solid with an isolated yield of 80% ¹H NMR (CDCl₃) δ 9.64 (s, 1H), 7.33 (d, J=3.7 Hz, 1H), 7.03 (s, 2H), 6.79 (d, J=3.7 Hz, 1H), 3.95 (s, 6H), 3.90 (s, 3H). ¹³C NMR (CDCl₃) δ 177.02, 159.41, 153.71, 151.90, 139.70, 124.45, 107.47, 102.69, 61.01, 56.35. HRMS (ESI) m/z: Calculated for C₁₄H₁₄O₅Na [M+Na]⁺285.0739; Found 285.0739.

### 5-(4-(trifluoromethyl)phenyl)furan-2-carbaldehyde (2f)

White solid with an isolated yield of 75% ¹H NMR (CDCl₃) δ 9.73 (s, 1H), 7.96 (d, J=8.2 Hz, 2H), 7.73 (d, J=8.2 Hz, 2H), 7.37 (d, J=3.8 Hz, 1H), 6.98 (d, J=3.8 Hz, 1H). ¹³C NMR (CDCl₃) δ 177.46, 157.42, 152.59, 132.16, 132.14, 131.34, 131.02, 126.01, 125.97, 125.41, 123.06, 122.47, 109.25. HRMS (ESI) m/z: Calculated for C₁₂H₇F₃O₂Na [M+Na]⁺ 263.0297; Found 263.0296.

### Synthesis of 4′-fluoro-[1,1′-biphenyl]-3-carbaldehyde (2g)

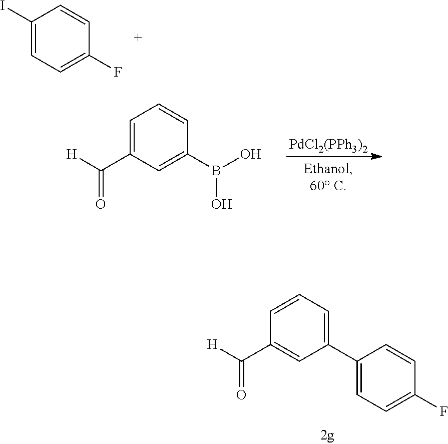 [SMILES: Fc1ccc(I)cc1 ; O=Cc1cccc(B(O)O)c1 ; O=Cc1cccc(-c2ccc(F)cc2)c1 ; "PdCl2(PPh3)2"]

1-fluoro-4-iodobenzene (1.35 mmol) and (3-formylphenyl)boronic acid (1.62 mmol) were added to a 20 ml vial, followed by K₂CO₃ (1.62 mmol) and 4 ml of solvent (DME/Ethanol/Water-1:0.5:1) was added under argon. Then, 0.07 mmol of PdCl₂(PPh₃)₂ was added and the mixture was stirred at 65° C. for about 4 h and stirred overnight at room temperature. At the consumption of the starting material, the solvent was removed in vacuo and the residue diluted with dichloromethane and washed successively with dilute hydrochloric acid (0.5M, 100 ml×3), saturated sodium bicarbonate (100 ml×3), brine (100×1) and the organic layer was dried and anhydrous sodium sulphate. The crude product was purified by silica gel column chromatography (DCM only) to afford a pure product 2g as white solid with an isolated yield of 62%. ¹H NMR (CDCl₃) δ 10.08 (s, 1H), 8.05 (t, J=1.7 Hz, 1H), 7.89-7.82 (m, 1H), 7.80 (dt, J=7.7, 1.5 Hz, 1H), 7.66-7.52 (m, 3H), 7.16 (t, J=8.6 Hz, 2H). ¹³C NMR (CDCl₃) δ 192.25, 164.08, 161.62, 141.10, 136.96, 135.82, 135.79, 132.85, 129.58, 129.03, 128.83, 128.77, 128.75, 127.81, 127.13, 116.04, 115.83. HRMS (ESI) m/z: Calculated for C₁₃H₉FONa [M+Na]⁺223.0524; Found 223.0535.

### General Procedure for the Synthesis of BW-PS-101-106

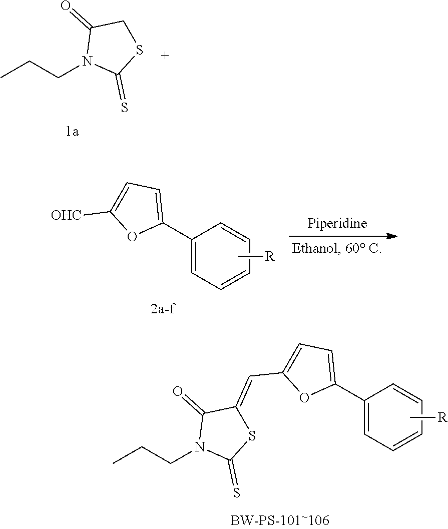

R: 101: H; 102:4-F; 103: 4-OH; 104:4-OCH₃; 105: 3,4,5-(OCH₃)₃; 106: 4-CF₃

Compound 2a-f (0.24 mmol), compound 1a (0.24 mmol), and 3 ml of ethanol were added to a 20 ml vial, followed by the addition of piperidine (0.24 mmol) under argon. The mixture was then stirred at 60° C. for about 4 h and stirred overnight at room temperature. At the consumption of the starting material determined by TLC, the solvent was removed in vacuo and the residue diluted with dichloromethane and washed successively with dilute hydrochloric acid (0.5M, 100 ml×3), saturated sodium bicarbonate (100 ml×3), brine (100×1) and the organic layer was dried and anhydrous sodium sulphate. The crude product was purified by silica gel column chromatography (DCM only) to afford a pure product BW-PS-101-106.

### (Z)-5-((5-phenylfuran-2-yl)methylene)-3-propyl-2-thioxothiazolidin-4-one (BW-PS-101)

Yellow solid with an isolated yield of 94%. ¹H NMR (CDCl₃) δ 7.81 (d, J=7.7 Hz, 2H), 7.50 (t, J=3.9 Hz, 3H), 7.42 (d, J=7.4 Hz, 1H), 6.97 (d, J=3.8 Hz, 1H), 6.89 (d, J=3.8 Hz, 1H), 4.12 (dd, J=8.7, 6.6 Hz, 2H), 1.79 (q, J=7.5 Hz, 2H), 1.01 (t, J=7.5 Hz, 3H). ¹³C NMR (CDCl₃) δ 194.60, 167.62, 158.87, 149.48, 129.27, 129.15, 128.98, 125.33, 124.74, 121.31, 120.01, 117.80, 108.92, 46.11, 20.49, 11.25. HRMS (ESI) m/z: Calculated for C₁₇H₁₅NO₂S₂Na [M+Na]⁺352.0445; Found 352.0442.

### (Z)-5-((5-(4-fluorophenyl)furan-2-yl)methylene)-3-propyl-2-thioxothiazolidin-4-one (BW-PS-102)

Yellow solid with an isolated yield of 79%. ¹H NMR (CDCl₃) δ 7.84-7.76 (m, 2H), 7.49 (s, 1H), 7.21 (t, J=8.6 Hz, 2H), 6.97 (d, J=3.7 Hz, 1H), 6.83 (d, J=3.7 Hz, 1H), 4.12 (dd, J=8.6, 6.7 Hz, 2H), 1.79 (q, J=7.5 Hz, 2H), 1.01 (t, J=7.5 Hz, 3H). ¹³C NMR (CDCl₃) δ 194.39, 167.89, 164.27, 161.94, 157.87, 149.44, 126.72, 126.64, 125.58, 121.28, 119.78, 117.70, 116.49, 116.27, 108.60, 46.12, 20.49, 11.25. HRMS (ESI) m/z: Calculated for C₁₇H₁₅FNO₂S₂ [M+H]⁺ 348.0538; Found 348.0528.

### (Z)-5-((5-(4-hydroxyphenyl)furan-2-yl)methylene)-3-propyl-2-thioxothiazolidin-4-one (BW-PS-103)

Red solid with an isolated yield of 80%. ¹H NMR (CDCl₃) δ 7.72 (d, J=8.7 Hz, 2H), 7.49 (s, 1H), 7.01-6.94 (m, 3H), 6.76 (d, J=3.7 Hz, 1H), 4.17-4.09 (m, 2H), 1.79 (q, J=7.5 Hz, 2H), 1.00 (t, J=7.5 Hz, 3H). ¹³C NMR (CDCl₃) δ 194.54, 167.59, 159.00, 156.52, 148.91, 126.67, 122.16, 121.81, 118.80, 117.95, 116.18, 107.57, 46.11, 20.49, 11.25. HRMS (ESI) m/z: Calculated for C₁₇H₁₄NO₃S₂ [M−H]⁻ 344.0416; Found 344.0415.

### (Z)-5-((5-(4-methoxyphenyl)furan-2-yl)methylene)-3-propyl-2-thioxothiazolidin-4-one (BW-PS-104)

Orange-red solid with an isolated yield of 84%. ¹H NMR (CDCl₃) δ 7.80-7.72 (m, 2H), 7.48 (s, 1H), 7.08-6.99 (m, 2H), 6.96 (d, J=3.7 Hz, 1H), 6.76 (d, J=3.7 Hz, 1H), 4.17-4.08 (m, 2H), 3.91 (s, 3H), 1.79 (q, J=7.5 Hz, 2H), 1.00 (t, J=7.4 Hz, 3H). ¹³C NMR (CDCl₃) δ 194.64, 167.65, 160.58, 159.24, 148.92, 126.44, 121.89, 121.81, 118.97, 117.92, 114.65, 107.56, 55.46, 46.08, 20.49, 11.25. HRMS (ESI) m/z: Calculated for C₁₈H₁₈NO₃S₂ [M+H]⁺ 360.0728; Found 360.0728.

### (Z)-3-propyl-2-thioxo-5-((5-(3,4,5-trimethoxyphenyl)furan-2-yl)methylene)thiazolidin-4-one (BW-PS-105)

Orange-red solid with an isolated yield of 92%. ¹H NMR (CDCl₃) δ 7.50 (s, 1H), 7.05 (s, 2H), 6.97 (d, J=3.7 Hz, 1H), 6.82 (d, J=3.7 Hz, 1H), 4.16-4.06 (m, 2H), 4.01 (s, 6H), 3.94 (s, 3H), 1.85-1.71 (m, 2H), 1.00 (t, J=7.4 Hz, 3H). ¹³C NMR (CDCl₃) δ 194.15, 167.58, 158.48, 153.79, 149.27, 139.29, 124.50, 121.18, 119.95, 117.67, 108.57, 102.07, 61.08, 56.31, 46.08, 20.49, 11.25. HRMS (ESI) m/z: Calculated for C₂₀H₂₁NO₅S₂Na [M+Na]⁺ 442.0745; Found 442.0759.

### (Z)-3-propyl-2-thioxo-5-((5-(4-(trifluoromethyl)phenyl)furan-2-yl)methylene)thiazolidin-4-one (BW-PS-106)

Yellow solid with an isolated yield of 73%. H NMR (CDCl₃) δ 7.91 (d, J=8.2 Hz, 2H), 7.76 (d, J=8.2 Hz, 2H), 7.52 (s, 1H), 6.99 (s, 2H), 4.17-4.09 (m, 2H), 1.79 (q, J=7.5 Hz, 2H), 1.01 (t, J=7.4 Hz, 3H). ¹³C NMR (CDCl₃) δ 194.19, 167.54, 156.83, 150.34, 132.12, 126.21, 126.17, 124.75, 121.33, 120.81, 117.38, 110.54, 46.16, 20.49, 11.26. HRMS (ESI) m/z: Calculated for C₁₈H₁₄F₃NO₂S₂Na [M+Na]⁺420.0316; Found 420.0316.

### Synthesis of (Z)-5-((4′-fluoro-[1,1′-biphenyl]-3-yl)methylene)-3-propyl-2-thioxothiazolidin-4-one (BW-PS-107)

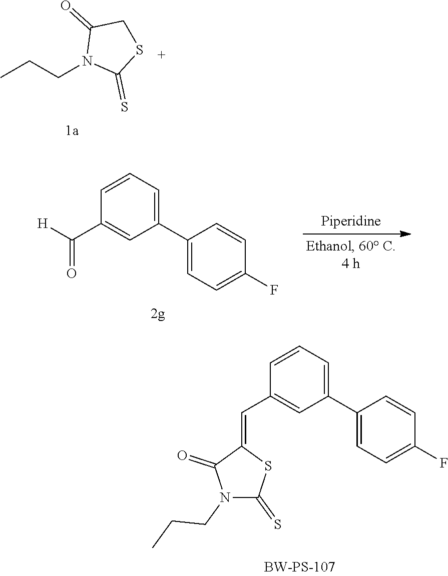

Compound 2g (0.24 mmol) and compound 1a (0.24 mmol) were added to a 20 ml vial, followed by piperidine (0.24 mmol) and 3 ml of ethanol was added under argon. The mixture was then stirred at 60° C. for about 4 h and stirred overnight at room temperature. At the consumption of the starting material, the solvent was removed in vacuo and the residue diluted with dichloromethane and washed successively with dilute hydrochloric acid (0.5M, 100 ml×3), saturated sodium bicarbonate (100 ml×3), brine (100 ml×1) and the organic layer was dried and anhydrous sodium sulfate. The crude product was purified by silica gel column chromatography (DCM only) to afford the product BW-PS-107 as a light yellow solid with an isolated yield of 81%. ¹H NMR (CDCl₃) δ 7.81 (s, 1H), 7.68-7.49 (m, 6H), 7.20 (t, J=8.6 Hz, 2H), 4.18-4.09 (m, 2H), 1.79 (q, J=7.5 Hz, 2H), 1.01 (t, J=7.4 Hz, 3H). ¹³C NMR (CDCl₃) δ 193.35, 167.82, 164.11, 161.65, 141.50, 136.03, 136.00, 134.03, 132.66, 129.86, 129.41, 129.24, 128.92, 128.83, 128.75, 123.70, 116.12, 115.90, 46.19, 20.45, 11.25. HRMS (ESI) m/z: Calculated for C₁₉H₁₇NOS₂ 358.0736 [M+H]⁺; Found 358.0747.

General Procedure for the synthesis of BW-PS-108˜110 and BW-PS-115˜120.

The synthesis procedure is slightly modified from the synthesis of BW-PS-101˜106. Specifically, Compound 2a, 2e, or 2f (0.2 mmol), compound 1b (0.2 mmol) or 3a˜e, was dissolved in 3 ml of ethanol in a 20 ml scintillation vial. To this solution piperidine (0.22 mmol) under argon. The mixture was then stirred at 60° C. for about 4 h. At the consumption of the starting material determined by TLC, the reaction was cooled to room temperature followed by crystallization in −15° C. freezer. Filtration followed by washing the solid with cold ethanol afforded the pure product BW-PS-108˜110 and BW-PS-115˜120.

### BW-PS-108

Yield: 63 mg (67%). ¹H NMR (400 MHz, CDCl₃) δ 7.79 (d, J=7.4 Hz, 2H), 7.48 (dd, J=9.1, 6.1 Hz, 3H), 7.39 (t, J=7.4 Hz, 1H), 6.96 (d, J=3.7 Hz, 1H), 6.87 (d, J=3.7 Hz, 1H), 4.31 (t, J=6.4 Hz, 2H), 3.71 (br-s, 4H), 2.77 (br-s, 2H), 2.62 (br-s, 4H). ¹³C NMR (101 MHz, CDCl₃) δ 194.62, 167.55, 158.99, 149.42, 129.34, 129.16, 128.93, 124.76, 121.56, 119.72, 118.06, 108.98, 77.35, 77.24, 77.04, 76.72, 66.71, 54.67, 53.63, 41.10.

### BW-PS-109

Yield: 87 mg (90%). ¹H NMR (400 MHz, CDCl₃) δ 7.88 (d, J=8.2 Hz, 2H), 7.73 (d, J=8.3 Hz, 2H), 7.49 (s, 1H), 6.97 (s, 2H), 4.32 (s, 2H), 3.72 (br-s, 4H), 2.77 (br-s, 2H), 2.61 (br-s, 4H). ¹³C NMR (101 MHz, CDCl₃) δ 194.23, 167.48, 156.93, 150.28, 132.07, 130.83, 130.46, 126.20, 126.17, 124.77, 122.53, 121.04, 117.67, 110.58, 66.60, 54.64, 53.57, 42.08.

### BW-PS-110

Orange solid, yield: 32 mg (78%). ¹H NMR (400 MHz, CDCl₃) δ 7.48 (s, 1H), 7.03 (s, 2H), 6.96 (d, J=3.7 Hz, 1H), 6.80 (d, J=3.7 Hz, 1H), 4.32 (br-s, 2H), 3.98 (s, 6H), 3.91 (s, 3H), 3.73 (br-s, 4H), 2.78 (br-s, 2H), 2.63 (br-s, 4H). ¹³C NMR (101 MHz, CDCl₃) δ 194.17, 167.51, 158.65, 153.80, 149.20, 139.38, 124.43, 121.48, 119.59, 117.99, 108.64, 102.14, 66.37, 61.08, 56.32, 54.61, 53.51, 29.72.

### BW-PS-115

Orange solid, yield: 63 mg (67%). ¹H NMR (400 MHz, Acetone) δ 8.10 (d, J=8.2 Hz, 2H), 7.89 (d, J=8.3 Hz, 2H), 7.61 (s, 1H), 7.39 (d, J=3.8 Hz, 1H), 7.31 (d, J=3.8 Hz, 1H), 4.23 (t, J=7.0 Hz, 2H), 3.54 (t, J=4.6 Hz, 4H), 2.43 (t, J=6.3 Hz, 2H), 2.32 (s, 4H), 1.97-1.87 (m, 2H). ¹³C NMR (101 MHz, Acetone) δ 194.33, 167.19, 156.48, 150.68, 132.64, 129.60, 126.23, 126.20, 124.96, 121.61, 121.21, 117.21, 111.57, 66.56, 56.22, 53.73, 43.29, 22.68.

### BW-PS-116

Orangish red solid, yield: 89 mg (99%). ¹H NMR (400 MHz, Acetone-d6) δ 8.11 (d, J=8.2 Hz, 2H), 7.90 (d, J=8.3 Hz, 2H), 7.63 (s, 1H), 7.41 (d, J=3.8 Hz, 1H), 7.33 (d, J=3.8 Hz, 1H), 4.15 (t, J=7.2, 2H), 3.59 (t, J=4.8, 4H), 2.42-2.29 (m, 6H), 1.78 (dt, J=14.5, 7.1 Hz, 2H), 1.54 (dt, J=14.5, 7.1 Hz, 2H).

### BW-PS-117

Orangish yellow solid, yield: 83 mg (90%). ¹H NMR (400 MHz, Acetone-d6) δ 8.12 (d, J=8.2 Hz, 2H), 7.91 (d, J=8.3 Hz, 2H), 7.64 (s, 1H), 7.41 (d, J=3.8 Hz, 1H), 7.34 (d, J=3.8 Hz, 1H), 4.24 (t, J=6.5 Hz, 2H), 2.80-2.75 (m, 4H), 2.72 (t, J=6.5 Hz, 2H), 2.59-2.52 (m, 4H). ¹³C NMR (101 MHz, Acetone-d6) δ 194.46, 167.00, 156.58, 150.70, 132.68, 129.98, 129.66, 126.19, 126.15, 125.62, 124.98, 122.92, 121.59, 120.89, 119.49, 117.39, 111.50, 55.16, 54.61, 41.67, 27.73.

### BW-PS-118

Orange solid, yield: 63 mg (67%).

### BW-PS-119

Orangish yellow solid, yield: 87 mg (90%). ¹H NMR (400 MHz, Acetone) δ 8.48 (d, J=3.6 Hz, 1H), 8.11 (d, J=8.1 Hz, 2H), 7.90 (d, J=8.2 Hz, 2H), 7.69 (t, J=6.9 Hz, 1H), 7.59 (s, 1H), 7.39 (d, J=3.5 Hz, 1H), 7.36-7.26 (m, 2H), 7.26-7.15 (m, 1H), 4.52 (t, J=7.5 Hz, 2H), 3.20 (t, J=7.5 Hz, 2H). ¹³C NMR (101 MHz, Acetone) δ 195.25, 167.73, 159.10, 157.50, 151.66, 150.27, 137.30, 133.66, 130.91, 130.59, 127.18, 127.14, 126.59, 125.94, 124.14, 123.89, 122.56, 122.54, 121.95, 118.24, 112.48, 44.86, 35.40.

### BW-PS-120

Orange solid, yield: 15 mg (75%). ¹H NMR (400 MHz, CDCl₃) δ 8.60 (d, J=4.4 Hz, 1H), 7.87 (t, J=7.2 Hz, 1H), 7.42 (d, J=3.9 Hz, 2H), 7.35 (d, J=7.9 Hz, 1H), 7.01 (s, 2H), 6.95 (d, J=3.7 Hz, 1H), 6.79 (d, J=3.7 Hz, 1H), 4.60 (t, J=7.0 Hz, 2H), 3.97 (s, 6H), 3.90 (s, 3H), 3.45 (t, J=6.9 Hz, 2H). ¹³C NMR (101 MHz, CDCl₃) δ 193.83, 167.15, 158.69, 156.33, 153.79, 149.12, 139.39, 131.13, 125.11, 124.41, 123.13, 121.62, 119.27, 118.09, 108.67, 102.14, 61.08, 60.92, 56.32, 43.77, 33.40.

### Example 2: SARS-CoV-2 Inhibition

The compounds described herein were tested for their abilities to inhibit SARS-CoV-2 infection. The antiviral work was conducted using SARS-CoV-2 infected Huh7 cells. The tested compounds and properties are shown in Table 3. Specifically, Table 3 displays the molecular weight (mw); the cLogP (the computationally calculated logarithm of the octanol-water partition coefficient); and the topological polar surface area (TPSA) for compounds as described herein. Compound 136, a known viral entry inhibitor developed for influenza (see below Table 3 for structure), was also tested.

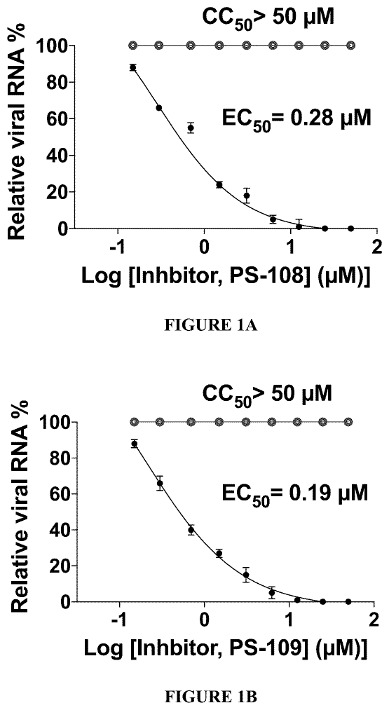

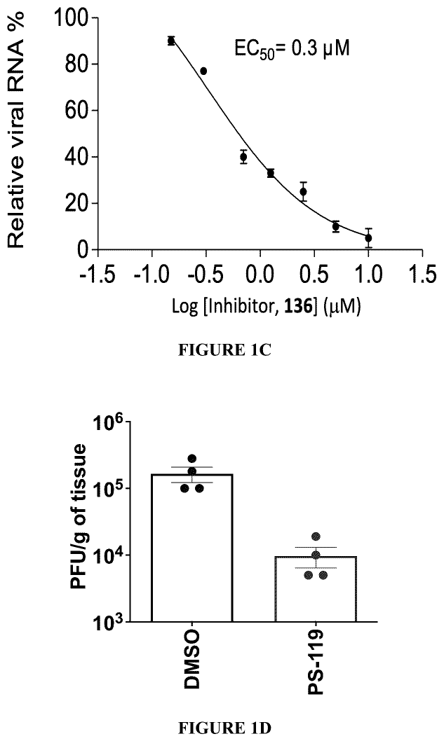

As shown in FIGS. 1A-1C, the compounds described herein showed significant inhibition of viral replication. BW-PS-108 and BW-PS-109 showed significant levels of inhibition of viral replication against SARS-COV-2 with EC₅₀ values of 280 and 190 nM, respectively. The results show that introducing a polar or ionizable group to the core scaffold (e.g., an ionizable morpholine group) as designed in the compounds described herein improves water solubility without the consequence of abolishing activity against the SARS-CoV-2 virus. No solubility issues were encountered when studying their toxicity at 100 μM.

The potency levels of the compounds described herein compare favorably against remdesivir and molnupiravir. In the same cell culture model as that used in the assay described herein, remdesivir, an FDA-approved anti-viral against SARS-COV-2, had an EC₅₀ of 700 nM. The potency level is also comparable to that of molnupiravir generated in similar cell culture studies. Further, the potency levels of the compounds described herein were also higher than Compound 136.

TABLE 3

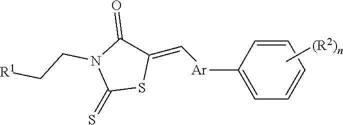

| BW-PS #. | R¹ | Ar | R² | Properties |
|---|---|---|---|---|
| 108 | N-morpholinyl | 2,5-furanyl | H | MW: 400, cLogP: 3.341, |
| | | | | tPSA: 42.01 |
| 109 | N-morpholinyl | 2,5-furanyl | 4-CF₃ | MW: 468, cLogP: 4.224, |
| | | | | tPSA: 42.01 |
| 119 | 2-pyridinyl | 2,5-furanyl | 4-CF₃ | MW: 460, cLogP: 4.895, |
| | | | | tPSA: 41.9 |

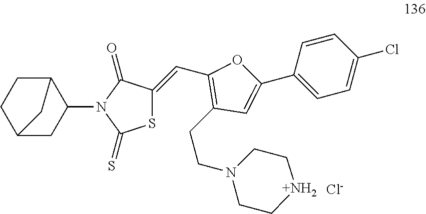 [SMILES: O=C1/C(=C/c2oc(-c3ccc(Cl)cc3)cc2CCN2CC[NH2+]CC2)SC(=S)N1C1CC2CCC1C2 ; [Cl-]]

The in vivo antiviral activities of the compounds described herein was also assessed. Six-week-old C57BL/6 mice were inoculated intranasally with 10⁵ plaque-forming units (PFU) of mouse-adapted SARS-CoV-2. Animals were injected with vehicle (DMSO) or 10 mg/kg of compound (e.g., BW-PS-119) intraperitoneally on days 0, 1, and 2 post infection. Animals were euthanized at three days after infection and lungs were collected. Virus titers were analyzed in the lungs by plaque assay. See FIG. 1D. In FIG. 1D, the data are expressed as PFU/g of tissue. Each data point represents an individual mouse. One log reduction in lung virus titers was observed.

In the mouse model of SARS-CoV-2 infection, BW-PS-119 was shown to be efficacious against mouse-adapted SARS-COV-2 (see FIG. 1D). In addition, BW-PS-119 enriched in the endosome, which was confirmed by imaging studies.

### Example 3: Cytotoxicity Evaluation

Photodynamic therapy (PDT) has been developed to treat cancer for more than 50 years. As a minimally invasive therapy, PDT has been receiving increasing attention in both cancer research and clinical applications over the past decades. PDT is based on the process of photoexcitation of a light-absorbing molecule, termed as the photosensitizer (PS), to its excited states. It can undergo either Type I photochemical electron transfer reaction to produce reactive oxygen species (ROS) such as hydroxyl radical from oxygen, or through Type II photochemical process to transfer its energy to ground triplet state oxygen to generate cytotoxic singlet oxygen (¹O₂). The illuminated cells, therefore, undergo cell apoptosis due to intense oxidative stress. Compared with other non-invasive cancer therapy such as radiotherapy, PDT features high selectivity, low repeat dosage induced resistance, low overdose exposure toxicity, fast healing, and outpatient compatibility. It is applied in treating solid tumors, including skin, lung, esophagus, bladder, ovarian, cervix, breast, pancreas, and prostate, as well as other non-oncological diseases.

### Methods

HeLa cells and MCF-7 cells were purchased from ATCC (Manassas, VA). HeLa cells were cultured in high glucose DMEM culture medium (Sigma-Aldrich; St. Louis, MO) with glutamine, without sodium pyruvate, and supplemented with 10% fetal bovine serum (FBS) and 1% penicillin and streptavidin. MCF-7 cells were cultured in MEM culture medium (Sigma-Aldrich) supplemented with 10% FBS and 1% penicillin and streptavidin. Cells were incubated in 37° C. incubator with 5% CO₂. Under dim light, compounds were dissolved in DMSO to form 20 mM stock solution and serially diluted in 96-well plate with full culture medium to form the working solution with DMSO concentration being 0.5% in all wells. HeLa and MCF-7 cells were seeded in 96-well plate at a density of 10⁵/100 μL/well and incubated overnight, and then the culture medium was replaced with 100 μL of the compound-loaded working solution. The dark treatment group was kept in the incubator for 24 hours. The exposure treatment group was allowed to incubate for 30 minutes, followed by exposure with blue light for the designated time (5 min for 0.45 J/cm²). After exposure, cells were returned to the incubator and incubated for 24 hours. 10 μL CCK-8 solution (Dojindo, Japan) was added to the cells in each well and incubated for 2 hours. The optical density at 450 nm of each well was read with the Perkin-Elmer Vector 3 plate reader (USA). Cells treated with 0.5% DMSO were used as the vehicle control and 100 μL culture medium with 10 μL CCK-8 was used as the blank control. Cell viability was calculated as:

$$Cell viability = \frac{OD 450 of treated well - OD 450 of blank control}{OD 450 of vehicle control - OD 450 of blank blank control} \times 100 \%$$

All tests were in triplicate, and the IC₅₀ was calculated with non-linear regression of concentration and cell viability by Graphpad Prism.

### Discussion

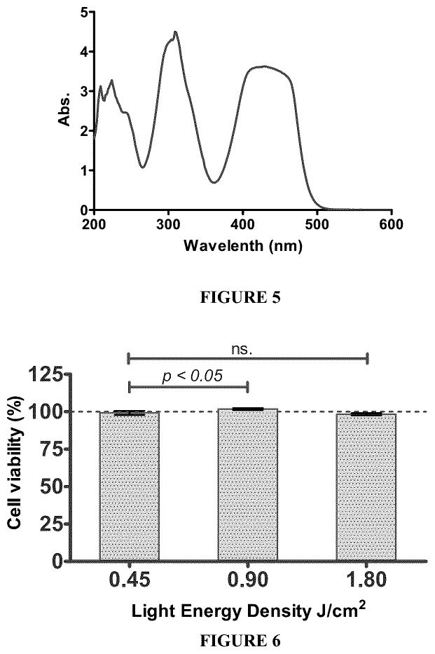

The compound BW-PS-101 showed an absorption peak at 410-465 nm, corresponding to the n-π* of the C═S bond (FIG. 5). Therefore, a low power blue LED light was used to conduct light-induced cytotoxicity study. BW-PS-101 was incubated with cervical carcinoma HeLa cell line under darkness. After incubation for 30 min to facilitate internalization, cells were exposed to a low-power blue LED array (470 nm, 1.5 mW/cm², as described above) for 5 minutes (total irradiation energy 0.45 J/cm²) and incubated for an additional 24 hours under darkness followed by cell viability determination with CCK-8 assay.

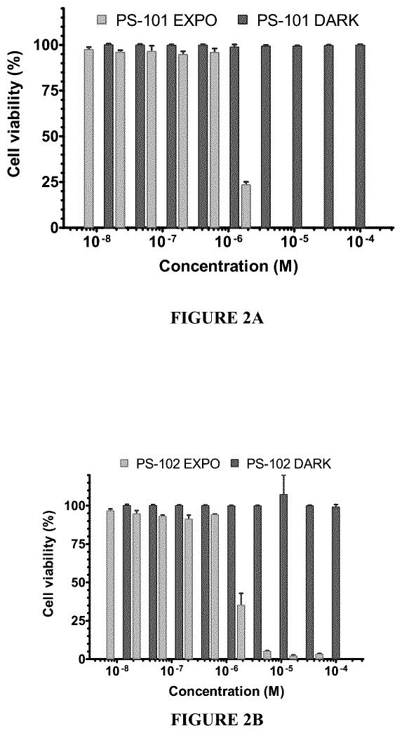

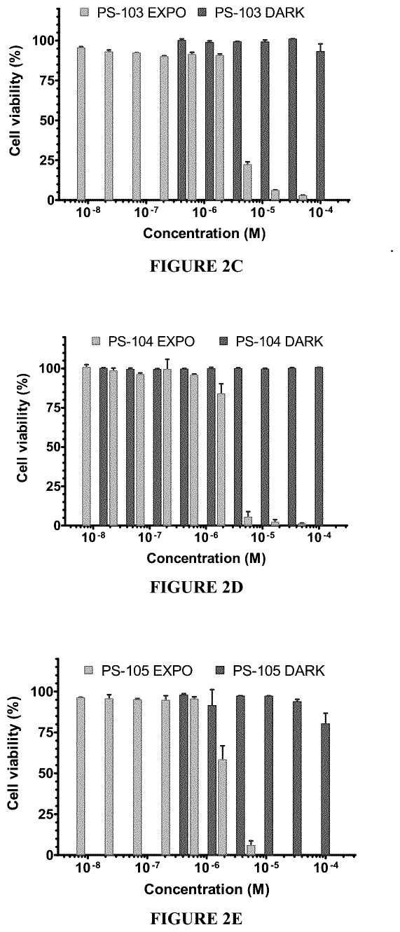

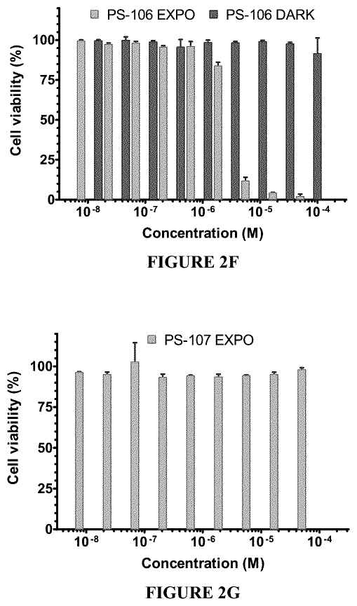

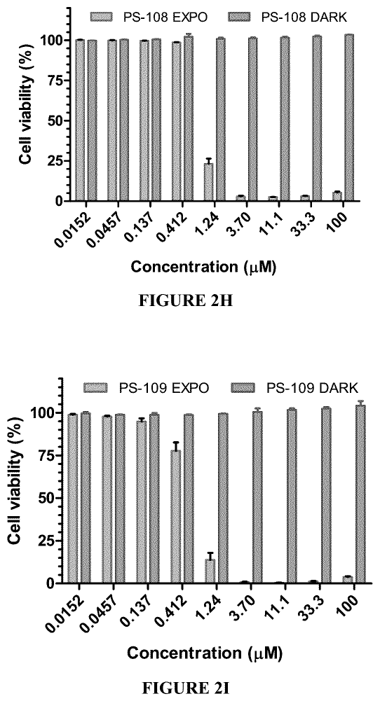

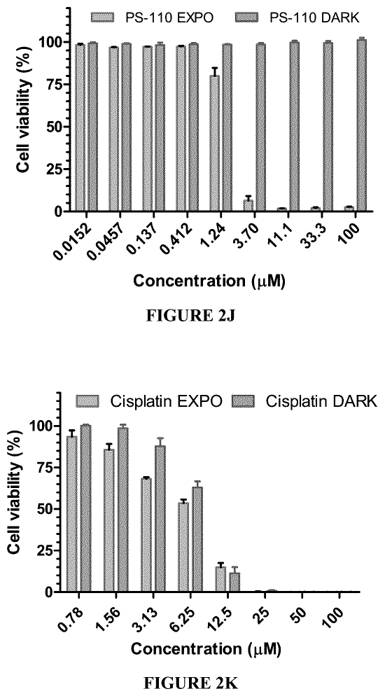

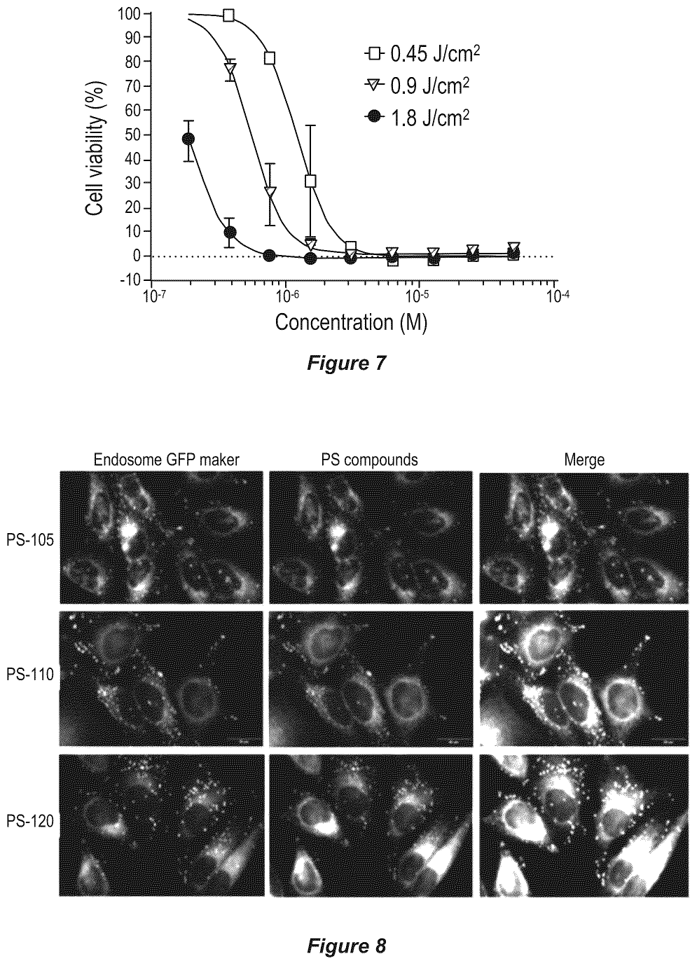

Cells treated in the dark did not show any cytotoxicity up to 100 μM of PS-101. Nevertheless, significant light-induced cytotoxicity was seen in the exposed cells with an IC₅₀ of 1.38±0.05 μM (FIG. 2A). Blue light irradiation alone did not affect cell viability (FIG. 4). To further verify if the cytotoxicity is light-dependent, cells were incubated with different concentrations of the BW-PS-101 and exposed to blue light with different duration (5, 10, and 20 min), thus resulted in different irradiation energy density (0.45-1.8 J/cm²). Results shown in FIG. 7 indicate an explicit light energy dependency of the light-induced cytotoxicity. As a reference, the cell viability was not affected by the increased light energy (FIG. 6). It should be noted when the light energy went up to 1.8 J/cm², IC₅₀ in the HeLa cell reached the submicromolar range.

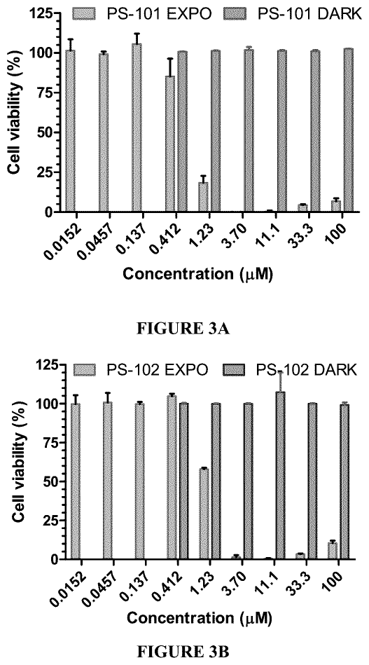

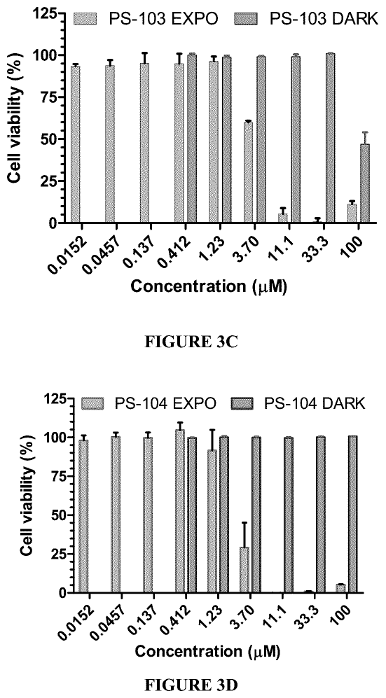

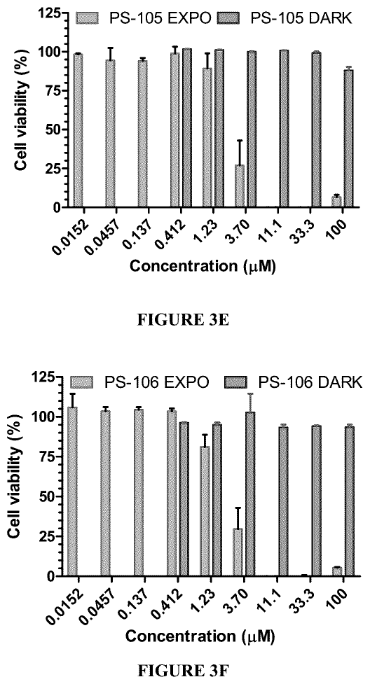

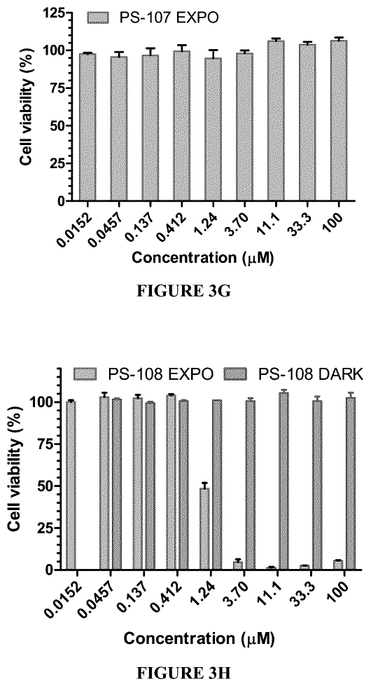

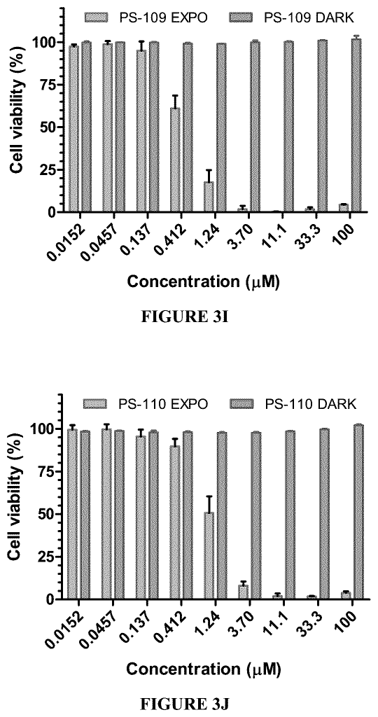

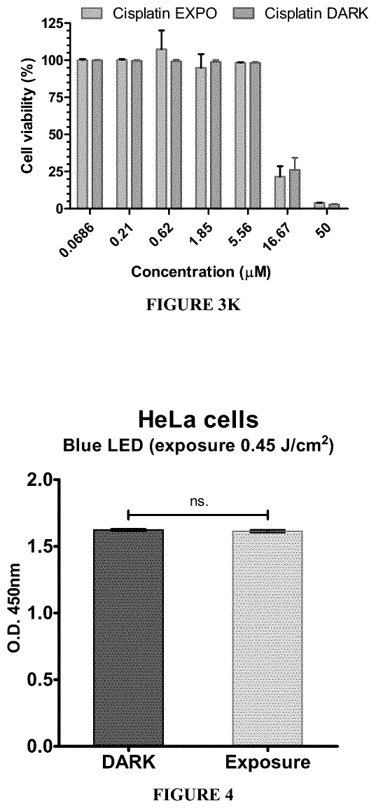

Additional compounds as described herein were tested. UV spectroscopy showed electron-donating groups such as hydroxyl and methoxyl red-shifted the E-band while electro-withdrawing groups such as fluoro- and trifluoromethyl group blue-shifted. Light-induced cytotoxicity was then evaluated. As shown in Table 4, fluoride derivative BW-PS-102 gave a comparable activity as BW-PS-101. PS-107, a compound with the furan ring replaced by phenyl, was synthesized as the structural mimetic control. PS-107 did not show any photo-induced cytotoxicity, indicating the furan ring is essential for the photo-induced cytotoxicity. As a positive control, cisplatin showed similar cytotoxicity with or without light irradiation. All compounds were also tested against MCF-7 breast cancer cell line, which gave a similar trend of activity (Table 4). The HeLa cell data for the compounds are shown in FIGS. 2A-2K and the MCF-7 cell data for the compounds are shown in FIGS. 3A-3K.

TABLE 4
Result of BW-PS compounds

| | Hela cells ^{a} | | MCF-7 cells ^{a} | | | |
|---|---|---|---|---|---|---|
| | Exposure | | Exposure | | | |
| PS | IC₅₀ | Dark IC₅₀ | IC₅₀ | Dark IC₅₀ | ϕ_{Δ} | Hammett |
| Compound | (μM) | (μM) | (μM) | (μM) | MeOH) | Constant |
| 101 | 1.38 ± 0.05 | >100 | 0.74 ± 0.14 | >100 | 0.109 | 0 |
| 102 | 1.48 ± 0.14 | >100 | 1.26 ± 0.004 | >100 | 0.075 | 0.06 |
| 103 | 3.58 ± 0.13 | >100 | 4.25 ± 0.17 | ≈100 | 0.007 | −0.37 |
| 104 | 2.71 ± 0.23 | >100 | 2.87 ± 0.92 | >100 | 0.015 | −0.27 |
| 105 | 2.13 ± 0.30 | >100 | 2.71 ± 0.78 | >100 | 0.028 | N/A |
| 106 | 2.96 ± 0.09 | >100 | 2.58 ± 0.73 | >100 | 0.182 | 0.54 |
| 107 | >100 | N/A | >100 | N/A | N/A | N/A |
| 108 | 0.95 ± 0.02 | >100 | 1.22 ± 0.01 | >100 | 0.086 | |
| 109 | 0.63 ± 0.07 | >100 | 0.53 ± 0.09 | >100 | 0.115 | |
| 110 | 1.74 ± 0.16 | >100 | 1.25 ± 0.17 | >100 | N/A | |
| Cisplatin | 5.95 ± 0.17 | 7.54 ± 0.45 | 13.28 ± 1.45 | 12.5 ± 2.86 | | |

^{a} Exposure light energy density 0.45 J/cm², result shown in mean ± SD.

### Example 4: Fluorescence Microscopy

Fluorescence microscopy showed an accumulation of fluorescent analogs BW-PS-105, BW-PS-110, and BW-PS-120 in early endosomes (FIG. 8). The trimethoxy substituted analog BW-PS-105, which was fluorescent with an Ex/Em wavelength of 491 nm/574 nm, was incubated with HeLa cell at a concentration of 1 μM. Fluorescent microscopy showed a fluorescent signal localized at the endosome. BW-PS-110 and BW-PS-120, with morpholine or pyridine substitution of rhodanine, respectively, showed good co-localization with the endosome marker, CellularLight endosomes-GFP vector. Endosome enrichment of the photosensitizer led to the potency increase of about 5 folds. Targeting photosensitizers to the endosome efficiently induces cancer cell apoptosis by photodynamic therapy. As the compounds (e.g., BW-PS-110 and BW-PS-120) showed excellent colocalization with the commercially available bioengineered fusion protein probe, the compounds themselves serves as a good endosome fluorescent tracker.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula: 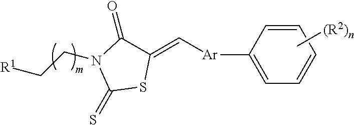
or a pharmaceutically acceptable salt or prodrug thereof, wherein:
m is 1, 2, 3, 4, or 5;
n is 0, 1, 2, 3, 4, or 5;
Ar is substituted or unsubstituted aryl or substituted or unsubstituted heteroaryl;
R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
each R² is independently halogen, hydroxyl, trifluoroalkyl, substituted or unsubstituted amino, substituted or unsubstituted alkoxy, or substituted or unsubstituted alkyl.

2. The compound of claim 1, wherein Ar is unsubstituted aryl or unsubstituted heteroaryl.

3. The compound of claim 1, wherein Ar is phenyl or furanyl.

4. The compound of claim 1, wherein R¹ is methyl, morpholinyl, morpholinylmethyl, morpholinylethyl, thiomorpholinyl, pyridinyl, or pyridinylmethyl.

5. The compound of claim 1, wherein n is 1, 2, 3, 4, or 5.

6. The compound of claim 1, wherein each R² is independently selected from the group consisting of fluoro, hydroxyl, alkoxy, and trifluoromethyl.

7. The compound of claim 1, wherein n is 0.

8. The compound of claim 1, wherein m is 1.

9. The compound of claim 1, wherein the compound is selected from the group consisting of:

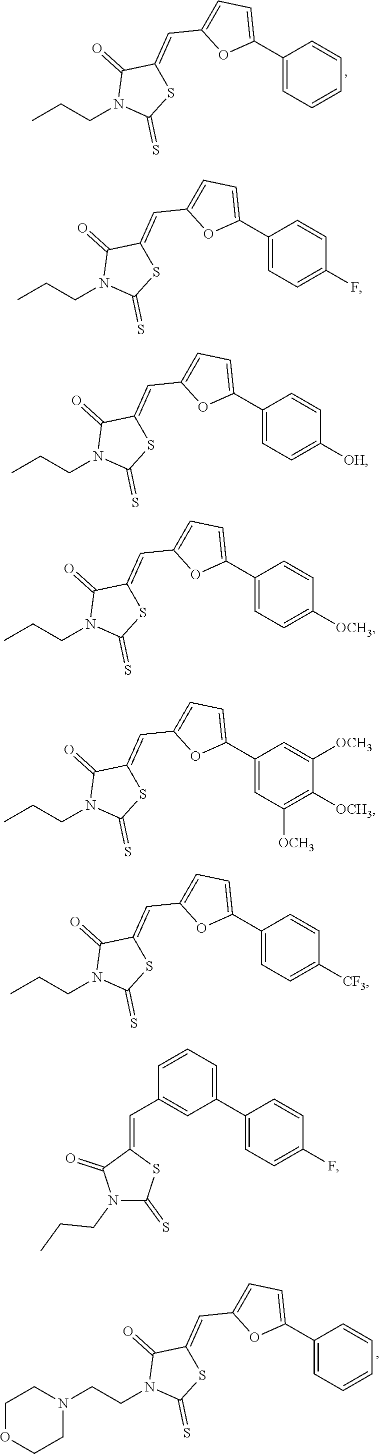

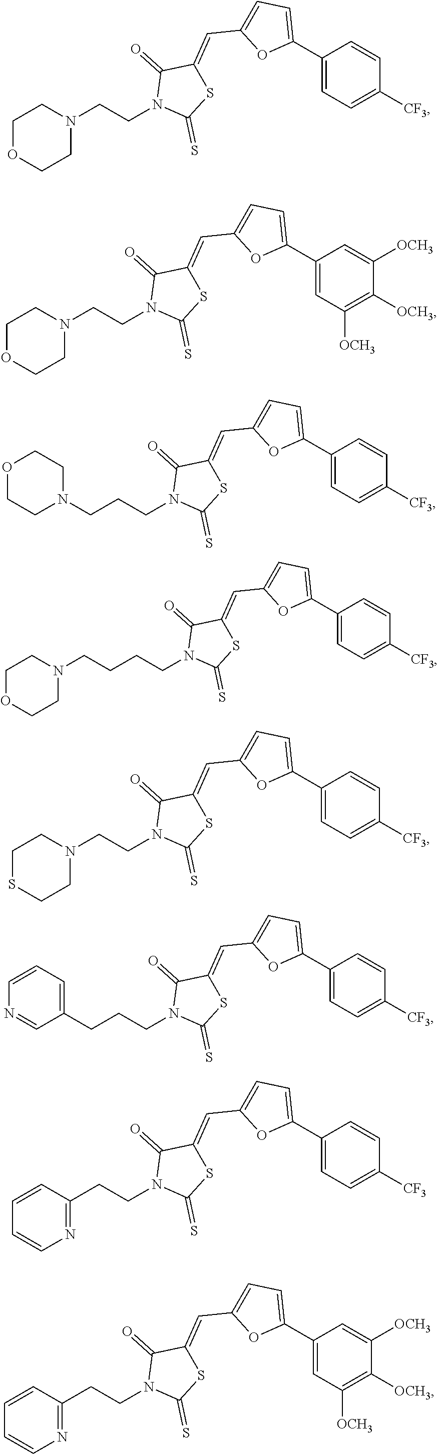

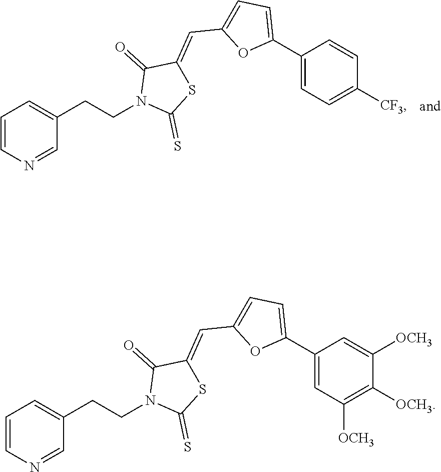 [SMILES: O=C1/C(=C/c2ccc(-c3ccc(C(F)(F)F)cc3)o2)SC(=S)N1CCc1cccnc1 ; COc1cc(-c2ccc(/C=C3\SC(=S)N(CCc4cccnc4)C3=O)o2)cc(OC)c1OC]

10. A pharmaceutical composition comprising a compound of claim 1 and a pharmaceutically acceptable carrier.

11. A kit comprising a compound of claim 1.

12. A method of treating or preventing a viral infection in a subject, comprising administering to the subject an effective amount of a compound of claim 1.

13. The method of claim 12, wherein the viral infection is a coronavirus infection.

14. The method of claim 13, wherein the coronavirus infection is a SARS-CoV-2 virus infection.

15. The method of claim 12, further comprising administering a second therapeutic agent to the subject.

16. The method of claim 15, wherein the second therapeutic agent is an antiviral compound.

17. A method of inhibiting viral replication in a cell, comprising contacting a cell with an effective amount of a compound of claim 1.

18. The method of claim 17, wherein the cell is infected with a coronavirus.

19. The method of claim 18, wherein the coronavirus is a SARS-CoV-2 virus.

20. The method of claim 17, wherein the contacting is performed in vitro or in vivo.

21. A method of treating or preventing cancer in a subject, comprising:
administering to the subject an effective amount of a compound of claim 1; and
applying energy to the subject at a wavelength capable of activating the compound or the pharmaceutical composition.
